(19) United States
(12) Patent Application Publication
(10) Pub. No.: US 2025/0304946 A1
(43) Pub. Date: Oct. 2, 2025

(54) IMMOBILIZED ENZYME NANOGEL AND PREPARATION METHOD AND APPLICATION THEREOF

(71) Applicants: SHANDONG UNIVERSITY, Jinan (CN); Shandong Tianhu Biological Technology Co., Ltd., Jinan (CN)

(72) Inventors: Yuxia Luan, Jinan (CN); Zhipeng Zhao, Jinan (CN); Qian Li, Jinan (CN); Yue Jiang, Jinan (CN); Hengliang Gao, Jinan (CN)

(73) Assignees: SHANDONG UNIVERSITY, Jinan (CN); Shandong Tianhu Biological Technology Co., Ltd., Jinan (CN)

(21) Appl. No.: 18/619,282

(22) Filed: Mar. 28, 2024

(30) Foreign Application Priority Data
Apr. 10, 2023 (CN) 202310398500.4

(51) Int. Cl. C12N 11/10; C12N 9/02; C12N 9/04; C12N 9/10; C12N 9/22; C12N 9/26; C12N 9/36; C12N 9/64; C12N 9/76; C12N 9/78

(52) U.S. Cl. CPC C12N 11/10; C12N 9/0006; C12N 9/0008; C12N 9/1025; C12N 9/22; C12N 9/2462; C12N 9/2474; C12N 9/641; C12N 9/6427; C12N 9/6491; C12N 9/78; C12Y 101/01001; C12Y 101/01027; C12Y 101/03004; C12Y 102/01003; C12Y 203/00; C12Y 301/21001; C12Y 304/21064; C12Y 304/22002; C12Y 305/04004

(57) ABSTRACT

A method for preparing and applying immobilized enzyme nanogels includes oxidized sodium alginate and enzymes are dissolved separately in deionized water, mixed uniformly, and reacted to obtain immobilized enzyme nanogels; the aldehyde groups in oxidized sodium alginate react with the amino groups in the enzymes, forming imine bonds to immobilize the enzymes; forming nano-scale cross-linked polymer-based particles with a three-dimensional network structure, resulting in the immobilized enzyme nanogels; the surface of immobilized collagenase nanogels is modified with CXCR4 antagonist peptides, which can specifically block CXCR4 on the surface of T cells; the Schiff base bonds can selectively break in the acidic microenvironment of tumors, leading to collagen degradation and reduced extracellular matrix density; the modification enhances the chemotaxis and infiltration of T cells into pancreatic cancer tissues and can inhibit the metastasis of pancreatic cancer.

### CROSS-REFERENCE TO RELATED APPLICATIONS

This application claims priority to Chinese Patent Application Ser. No. CN202310398500.4 filed on 10 Apr. 2023.

### TECHNICAL FIELD

This invention belongs to the field of medical technology, specifically involving an immobilized enzyme nanogel and preparation method and application thereof.

### BACKGROUND OF THE INVENTION

The disclosure of information in this section is solely intended to enhance the overall understanding of the background of the present invention, and should not necessarily be construed as an acknowledgment or in any way imply that such information constitutes prior art known to those skilled in the art

Enzymes, as biocatalysts, have advantages such as mild reaction conditions, high selectivity, high specificity, and high conversion rates, making them promising for applications. However, due to their inherent low stability, enzymes are prone to inactivation in the environment, limiting their practical application. Currently, immobilizing enzymes through physical or chemical methods is an effective strategy to enhance enzyme stability. Some preparation methods for immobilized enzymes have been disclosed in the prior art, along with applications in catalyzing industrial production and wastewater treatment.

However, due to the complexity and specificity of organism structures, as well as the safety of the materials and processes involved in the preparation of currently disclosed immobilized enzymes, and issues such as mismatched size and performance of immobilized enzymes with organisms, the currently available immobilized enzymes are not suitable for in vivo applications. Therefore, there is an urgent need to develop a new method for immobilizing enzymes suitable for the field of biopharmaceuticals.

Nanogels are porous polymer colloidal networks formed through physical or chemical cross-linking, offering chemical and mechanical stability and enabling efficient drug loading, making them excellent drug carriers. The use of chemically cross-linked immobilized enzyme nanogels holds promise for broader applications of enzyme-based drugs in the field of biopharmaceuticals. However, this technology faces significant challenges: on the one hand, due to the enzyme's susceptibility to inactivation, the preparation conditions for enzyme-based nanogels must be mild, avoiding harsh reactions such as heating, ultrasound, and the use of common reaction media like organic solvents, strong acids, and bases. On the other hand, with a focus on biomedical applications, the preparation of enzyme-based nanogels should minimize the introduction of toxic reagents and catalysts to ensure the safety of biological applications. These technical issues limit the development of enzyme-based nanogels.

Pancreatic cancer is one of the most challenging malignant tumors, often referred to as the “king of cancers”. The emergence of immunotherapy has brought hope to many cancer patients, but immunotherapy for pancreatic cancer remains one of the toughest challenges. T cells are the main attacking immune cells of the immune system against tumors, but the unique tumor microenvironment in pancreatic cancer greatly limits the effectiveness of T cells. Firstly, the extracellular matrix (ECM) in pancreatic cancer, primarily composed of collagen, is extremely dense, making it difficult for immune cells to infiltrate the tumor tissue. Secondly, in pancreatic cancer tissue, cancer-associated fibroblasts (CAFs) secrete the chemokine CXCL12, which can specifically bind to the chemokine receptor CXCR4 on the surface of T cells, restricting T cell movement and hindering their contact with tumor cells. Therefore, various immunotherapies, including immune checkpoint blockade therapy, have not shown significant efficacy. Targeting the elimination of ECM is a promising treatment strategy that can enhance the infiltration of drugs and immune cells into tumors. However, ECM elimination may accelerate the metastasis of pancreatic cancer. Therefore, the key to improving immunotherapy for pancreatic cancer lies in increasing T cell infiltration into the tumor and enhancing the contact between T cells and tumor cells while avoiding tumor metastasis.

### SUMMARY OF THE INVENTION

To overcome the above issues, the present invention provides a method for immobilized enzyme nanogels and their preparation and application.

To achieve the above technical objectives, the present invention adopts the following technical solution:

In a first aspect of the present invention, a method for preparing immobilized enzyme nanogels is disclosed, comprising:

Oxidized sodium alginate and enzymes are separately dissolved in deionized water, mixed uniformly, and then reacted to obtain immobilized enzyme nanogels.

The aldehyde groups in oxidized sodium alginate react with the amino groups in the enzyme via a Schiff base reaction, forming an imine bond. This Schiff base reaction does not require additional conditions such as heating or catalysts; it can proceed in an aqueous solution. Therefore, this reaction is suitable for synthesizing immobilized enzymes to prevent enzyme inactivation. By using multiple imine bonds to link oxidized sodium alginate and the enzyme together, the enzyme's conformation becomes relatively stable. Since oxidized sodium alginate is a long-chain polymer, enzymes in the solution undergo intermolecular crosslinking. This crosslinking creates a three-dimensional network structure where enzyme molecules are immobilized at the crosslinking points of the polymer. The distances between these crosslinking points are within the nanometer scale, forming nano-scale oligomers. As a result, the immobilized enzyme obtained forms a gel structure at the nanometer scale, known as enzyme nanogels.

The enzymes described in the present invention are derived from biological organisms, possess catalytic functions, contain no less than two amino functional groups, and have a chemical composition that consists of protein components.

Furthermore, the enzymes include: (1) oxidoreductases, such as dehydrogenases, oxidases, reductases, peroxidases; (2) transferases, such as methyltransferases, aminotransferases, proteases, lipases, polymerases; (3) hydrolases, such as amylases; (4) lyases, such as dehydratases, decarboxylases; (5) isomerases, such as mutases, isomerases; (6) synthetases, such as glutamine synthetase; (7) mutases, such as alcohol oxidases. Further preferred examples include: collagenase, trypsin, deoxyribonuclease I, proteinase K, lactate dehydrogenase, ethanol dehydrogenase, glutamine transaminase, aldehyde dehydrogenase, lysozyme, adenosine deaminase, papain, glucose oxidase, and hyaluronidase.

In a second aspect of the present invention, immobilized enzyme nanogels prepared by the above-mentioned method are disclosed.

In a third aspect of the present invention, a method for preparing immobilized matrix-degrading enzyme nanogels modified with CXCR4 antagonist peptides is disclosed, comprising:
(1) Oxidized sodium alginate and matrix-degrading enzymes are separately added to deionized water, mixed thoroughly, stirred and reacted in an ice bath for a period of time to obtain immobilized matrix-degrading enzyme nanogels;
(2) While stirring, CXCR4 antagonist peptides are added dropwise to the immobilized matrix-degrading enzyme nanogels prepared above. The reaction results in CXCR4 antagonist peptide-modified immobilized matrix-degrading enzyme nanogels.

In a fourth aspect of the present invention, CXCR4 antagonist peptide-modified immobilized matrix-degrading enzyme nanogels prepared by the above method are disclosed.

In a fifth aspect of the present invention, the application of the aforementioned CXCR4 antagonist peptide-modified immobilized matrix-degrading enzyme nanogels in the preparation of anti-tumor drugs is disclosed, with at least one or more of the following purposes:
1) Reduce the density of the extracellular matrix of tumor cells;
2) Increase the infiltration of lymphocytes into tumor;
3) Enhance the migration of T cells towards tumors;
4) Inhibit tumor metastasis;
5) Increase the effect of CAR-T cell solid tumor.
   Wherein, purposes 3), 4) and 5) are both related to CXCR4.

In a sixth aspect of the present invention, a composition of anti-tumor drugs is disclosed, wherein the composition comprises the CXCR4 antagonist peptide-modified immobilized matrix-degrading enzyme nanogels.

The beneficial effects of the present invention include:
(1) The aldehyde groups in oxidized sodium alginate react with the amino groups in the enzyme via a Schiff base reaction, forming an imine bond. This Schiff base reaction does not require additional conditions such as heating or catalysts; it can proceed in an aqueous solution. Therefore, this reaction is suitable for synthesizing immobilized enzymes to prevent enzyme inactivation. By using multiple imine bonds to link oxidized sodium alginate and the enzyme together, the conformation of the enzyme becomes relatively stable. Since oxidized sodium alginate is a long-chain polymer, enzymes in the solution undergo intermolecular crosslinking. This crosslinking creates a three-dimensional network structure where enzyme molecules are immobilized at the crosslinking points of the polymer. The distances between these crosslinking points are within the nanometer scale, forming nano-scale oligomers. As a result, the immobilized enzyme obtained forms a gel structure at the nanometer scale, known as enzyme nanogels. In this invention, nanoscale immobilized enzyme nanogels are obtained through chemical crosslinking, with the entire preparation process under mild reaction conditions to prevent enzyme inactivation. No toxic reagents are introduced, ensuring the safety of biological applications. The final synthesized immobilized enzyme nanogels are in nano size, making them easily utilized by the body.
(2) The invention uses one of the immobilized enzyme nanogels as an example to study its specific application. Through this specific application, it is demonstrated that the immobilized enzyme nanogels provided by this invention are suitable for use in the body. The invention surface-modifies the immobilized matrix-degrading enzyme nanogels with CXCR4 antagonist peptides, resulting in a CXCR4 antagonist peptide-modified immobilized matrix-degrading enzyme nanogel. This nanogel can specifically block CXCR4 on the surface of T cells. The crosslinked Schiff base bonds can responsively break in the acidic microenvironment of tumors, releasing collagenase and DV1 (a synthetic peptide composed of D-amino acids, with the peptide sequence LGASWHRPDKCCLGYQKRPLP), which degrade collagen, reduce ECM density, improve the chemotaxis and infiltration capabilities of T cells into pancreatic cancer tissues, effectively increase the number of lymphocytes infiltrating tumors, and inhibit the metastasis of pancreatic cancer.

### BRIEF DESCRIPTION OF DRAWINGS

The description and drawings that constitute a part of the present invention are used to provide a further understanding of the present invention. The illustrative embodiments of the present invention and their descriptions are used to explain the present invention and do not constitute an improper limitation of the present invention.

FIG. 1 shows the particle size diagram of immobilized trypsin nanogels in Example 1.

FIG. 2 shows the particle size diagram of immobilized deoxyribonuclease I nanogel.

FIG. 3 shows the particle size diagram of immobilized proteinase K nanogel.

FIG. 4 shows the particle size diagram of immobilized lactate dehydrogenase nanogel.

FIG. 5 shows the particle size diagram of immobilized ethanol dehydrogenase nanogel.

FIG. 6 shows the particle size diagram of immobilized glutamine transferase nanogel.

FIG. 7 shows the particle size diagram of immobilized aldehyde dehydrogenase nanogel.

FIG. 8 shows the particle size diagram of immobilized lysozyme nanogel.

FIG. 9 shows the particle size diagram of immobilized adenosine deaminase nanogel.

FIG. 10 shows the particle size diagram of immobilized papain protease nanogel.

FIG. 11 shows the particle size diagram of immobilized glucose oxidase nanogel.

FIG. 12 shows the particle size diagram of immobilized hyaluronidase nanogel.

FIG. 13 shows the transmission electron microscopy image of immobilized collagenase nanogel prepared in Example 13 of the present invention.

FIG. 14 shows the transmission electron microscopy image of D#O-Case prepared in Example 14 of the present invention.

FIG. 15 shows the experimental photo of inhibiting tumor cell migration in experimental Example 1 of the present invention.

FIG. 16 shows the tumor growth curve in experimental Example 2 of the present invention.

FIG. 17 shows the imaging of tumors after different treatment methods in experimental Example 3 of the present invention.

### DETAILED DESCRIPTION OF THE INVENTION

It should be noted that the following detailed descriptions are exemplary and are intended to provide further explanation of the present invention. Unless otherwise indicated, all technical and scientific terms used in the present invention have the same meanings as understood by those skilled in the art of the present invention.

It should be noted that the terms used here are solely for describing specific implementation methods and are not intended to limit the exemplary embodiments of the present invention. As used herein, unless otherwise specified in context, the singular form is intended to include the plural form. Furthermore, it should be understood that when the terms “comprising” and/or “including” are used in this specification, they indicate the presence of features, steps, operations, devices, components, and/or combinations thereof.

The first typical embodiment of the present invention provides a method for preparing immobilized enzyme nanogels, comprising:

Mix oxidized sodium alginate and enzyme separately dissolved in deionized water, react after thorough mixing to obtain immobilized enzyme nanogels.

The enzymes described in the present invention are substances derived from living organisms, possessing catalytic functions, containing no less than two amino functional groups, and having a chemical composition of protein components.

In one or more embodiments, the enzymes comprise: (1) oxidoreductases, such as dehydrogenases, oxidases, reductases, peroxidases; (2) transferases, such as methyltransferases, aminotransferases, proteases, lipases, polymerases; (3) hydrolases, such as amylases; (4) lyases, such as dehydratases, decarboxylases; (5) isomerases, such as racemases, isomerases; (6) synthetases, such as glutamine synthetase; (7) mutases, such as alcohol oxidases. Further preferred enzymes include: collagenase, trypsin, immobilized deoxyribonuclease I, proteinase K, lactate dehydrogenase, ethanol dehydrogenase, glutamine transferase, aldehyde dehydrogenase, lysozyme, adenosine deaminase, papain protease, glucose oxidase, and hyaluronidase.

In one or more embodiments, the method for preparing oxidized sodium alginate comprises: dissolving sodium alginate in deionized water, adding sodium periodate, stirring in the dark at room temperature, adding ethylene glycol for reaction, adding NaCl to the reaction system for ethanol extraction, redissolving in water for dialysis, and finally obtaining oxidized sodium alginate through freeze-drying.

The principle of this reaction: Sodium periodate oxidizes sodium alginate, causing the sugar ring of sodium alginate to break during the reaction process. The breakage occurs at the carbon-carbon single bond adjacent to the hydroxyl group, forming an aldehyde group at each end of the breakage, creating a local dialdehyde structure. The oxidation product has multiple local dialdehyde structures and also retains multiple normal sugar ring structures that have not been oxidatively cleaved. These two types of structures are randomly distributed in the product, linked by glycosidic bonds, resulting in a polysaccharide-like structure known as oxidized sodium alginate.

In one or more embodiments, the mass ratio of oxidized sodium alginate to enzyme is 0.01 to 50:1.

In one or more embodiments, the concentration of oxidized sodium alginate in deionized water is 1 to 50 mg mL⁻¹.

The second typical embodiment of the present invention provides immobilized enzyme nanogels prepared using the above-mentioned preparation method.

The third typical embodiment of the present invention provides a method for preparing immobilized matrix-degrading enzyme nanogels modified with CXCR4 antagonist peptides, comprising:
(1) Dissolve oxidized sodium alginate and matrix-degrading enzyme separately in deionized water, mix thoroughly, stir the reaction mixture in an ice bath for a period of time to obtain immobilized matrix-degrading enzyme nanogels;
(2) While stirring, add CXCR4 antagonist peptides dropwise to the immobilized matrix-degrading enzyme nanogels obtained in step (1), react to obtain CXCR4 antagonist peptide-modified immobilized matrix-degrading enzyme nanogels.

In one or more embodiments, the matrix-degrading enzyme comprises collagenase, papain, trypsin, tissue protease, hyaluronidase, heparinase, and DNAase, preferably collagenase I-V, further preferably collagenase IV.

In one or more embodiments, the CXCR4 antagonist peptide is DV1 (a synthetic peptide composed of D-amino acids, with the peptide sequence of LGASWHRPDKCCLGYQKRPLP) and its salt derivatives, Balixafortide and its salt derivatives, CTCE-9908 and its salt derivatives, FC131 and its salt derivatives, ALX 40-4C and its salt derivatives, Motixafortide and its salt derivatives, CTCE-0214 and its salt derivatives, TC14012 and its salt derivatives, ATI-2341 and its salt derivatives, preferably DV1.

In one or more embodiments, the concentration of matrix-degrading enzyme dissolved in deionized water in step (1) is 0.5 to 5 mg/mL, preferably 1.11 mg/mL.

In one or more embodiments, the mass ratio of matrix-degrading enzyme to oxidized sodium alginate in step (1) is 1 to 10:1, preferably 5:1.

In one or more embodiments, the temperature of the ice bath in step (1) is 0 to 4° C.

In one or more embodiments, the stirring reaction time in step (1) is 4 to 12 hours, preferably 5 hours.

In one or more embodiments, the concentration of CXCR4 antagonist peptide in step (2) is 0.5 to 5 mg/mL, preferably 1 mg/mL.

In one or more embodiments, the mass ratio of CXCR4 antagonist peptide to matrix-degrading enzyme in step (2) is 1:0.1 to 10, preferably 1:2.

In one or more embodiments, the reaction time in step (2) is 0.1 to 5 hours, preferably 1 hour.

In one or more embodiments, the reaction in step (2) is carried out in an ice bath with a temperature of 0 to 4° C.

The fourth typical embodiment of the present invention provides CXCR4 antagonist peptide-modified immobilized matrix-degrading enzyme nanogels prepared using the above-mentioned method.

The fifth typical embodiment of the present invention provides the application of the above CXCR4 antagonist peptide-modified immobilized matrix-degrading enzyme nanogels in the preparation of anti-tumor drugs, with at least one or more of the following purposes:
1) Reduce the density of the extracellular matrix in tumor;
2) Increase the number of infiltrating lymphocytes in tumors;
3) Promote the migration of T cells towards tumors;
4) Inhibit tumor metastasis;
5) Increase the effect of CAR-T cell solid tumor.

Among these, purposes 3), 4) and 5) are both related to CXCR4.

The sixth typical embodiment of the present invention provides a formulation of an anti-tumor drug, comprising the above-mentioned CXCR4 antagonist peptide-modified immobilized matrix-degrading enzyme nanogels.

In order to allow technical personnel in this field to have a clearer understanding of the technical solution of the present invention, the following will provide a detailed explanation of the technical solution of the present invention based on specific examples.

### Implementation Example 1

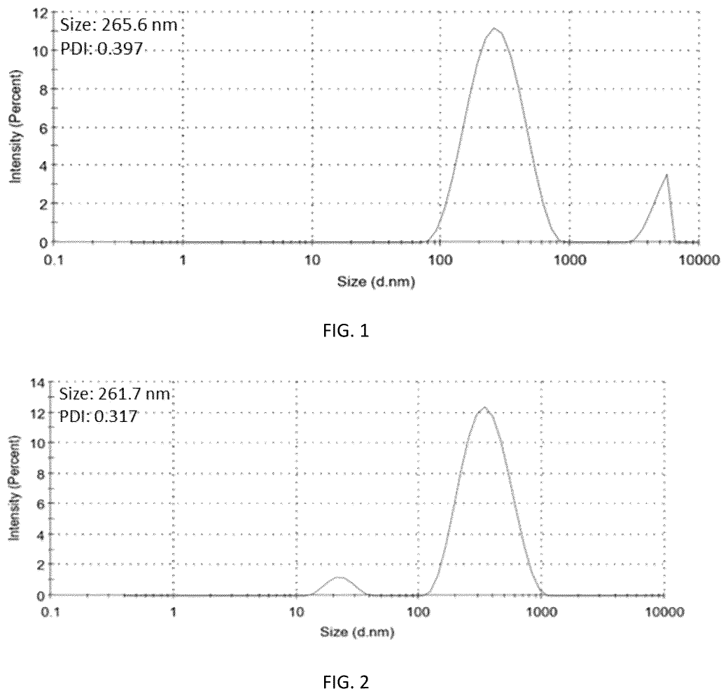

Preparation of immobilized trypsin nanogels: Trypsin and oxidized sodium alginate were separately dissolved in deionized water to prepare a 2.5 mg/mL trypsin solution and a 4.0 mg/mL oxidized sodium alginate solution. 0.8 mL of the trypsin solution and 0.1 mL of the oxidized sodium alginate solution were mixed in an ice bath and stirred magnetically for 5 hours to obtain immobilized trypsin nanogels. The particle size measurement results, as described in FIG. 1, show an average particle size of 265 nm.

### Implementation Example 2

Preparation of immobilized deoxyribonuclease I nanogels: Deoxyribonuclease I and oxidized sodium alginate were separately dissolved in deionized water to prepare a 1.25 mg/mL deoxyribonuclease I solution and a 1.0 mg/mL oxidized sodium alginate solution. 0.8 mL of the deoxyribonuclease I solution and 0.2 mL of the oxidized sodium alginate solution were mixed at room temperature and stirred magnetically for 30 minutes to obtain immobilized deoxyribonuclease I nanogels. The particle size measurement results, as described in FIG. 2, show an average particle size of 261 nm.

### Implementation Example 3

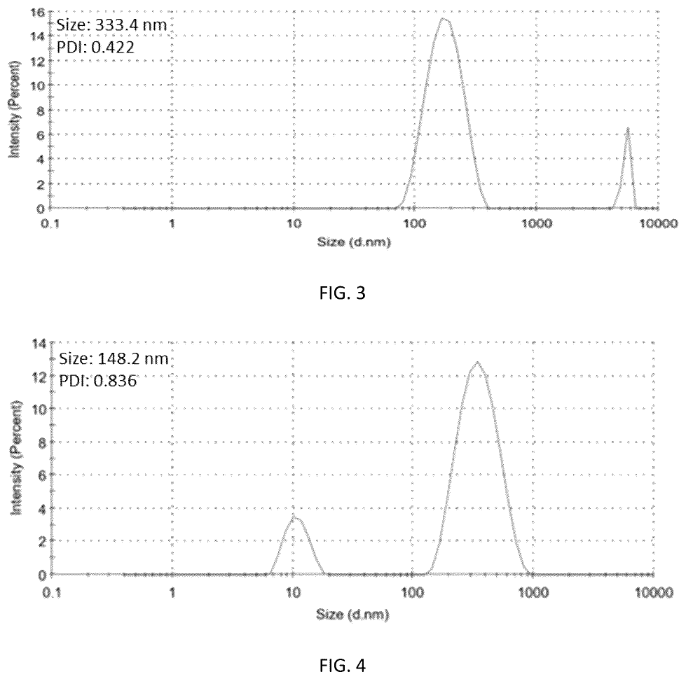

Preparation of immobilized proteinase K nanogel: Proteinase K and oxidized sodium alginate were dissolved in deionized water separately to prepare a 2.5 mg/mL proteinase K solution and a 1 mg/mL oxidized sodium alginate solution. 0.8 mL of the proteinase K solution and 0.2 mL of the oxidized sodium alginate solution were mixed under ice bath conditions, and the mixture was magnetically stirred for 5 hours to obtain the immobilized proteinase K nanogel. The particle size detection results, as described in FIG. 3, show that the average particle size is 333 nm.

### Implementation Example 4

Preparation of immobilized lactate dehydrogenase nanogel: Lactate dehydrogenase and oxidized sodium alginate were dissolved in deionized water separately to prepare a 2.0 mg/mL lactate dehydrogenase solution and a 2.0 mg/mL oxidized sodium alginate solution. 0.8 mL of the lactate dehydrogenase solution and 0.2 mL of the oxidized sodium alginate solution were mixed at room temperature and reacted for 1 hour to obtain the immobilized lactate dehydrogenase nanogel. The particle size detection results, as described in FIG. 4, show that the average particle size is 148 nm.

### Implementation Example 5

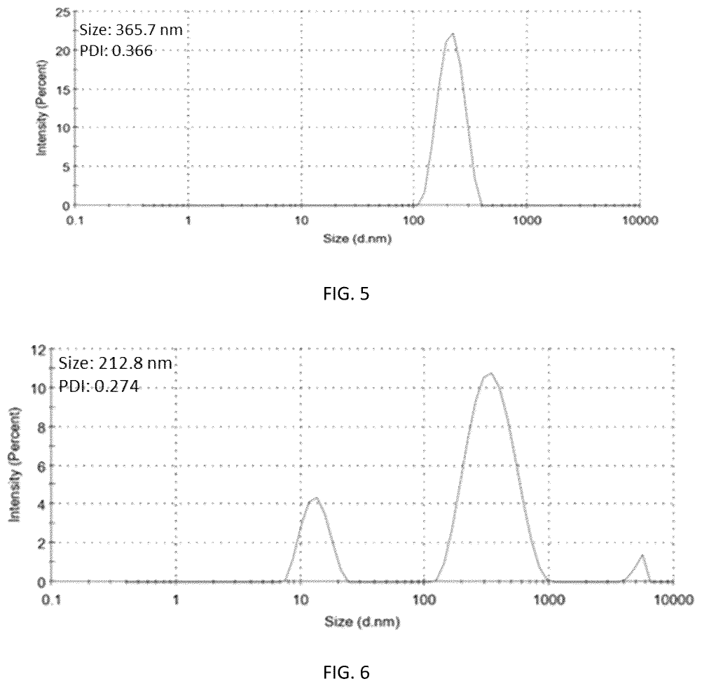

Preparation of immobilized alcohol dehydrogenase nanogel: Alcohol dehydrogenase and oxidized sodium alginate were dissolved in deionized water separately to prepare a 2.0 mg/mL alcohol dehydrogenase solution and a 1.0 mg/mL oxidized sodium alginate solution. 0.6 mL of the alcohol dehydrogenase solution and 0.4 mL of the oxidized sodium alginate solution were mixed at room temperature and reacted by shaking for 1 hour to obtain the immobilized alcohol dehydrogenase nanogel. The particle size detection results, as described in FIG. 5, show that the average particle size is 365 nm.

### Implementation Example 6

Preparation of immobilized glutamine transaminase nanogel: Glutamine transaminase and oxidized sodium alginate were dissolved in deionized water separately to prepare a 1.0 mg/mL glutamine transaminase solution and a 2.0 mg/mL oxidized sodium alginate solution. 0.8 mL of the glutamine transaminase solution and 0.2 mL of the oxidized sodium alginate solution were mixed at room temperature and reacted for 30 minutes to obtain the immobilized glutamine transaminase nanogel. The particle size detection results, as described in FIG. 6, show that the average particle size is 212 nm.

### Implementation Example 7

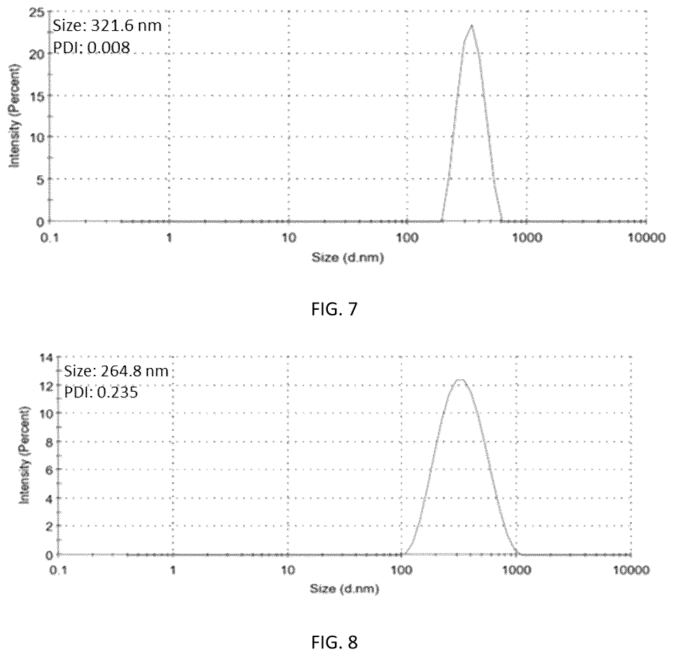

Preparation of immobilized aldehyde dehydrogenase nanogel: Aldehyde dehydrogenase and oxidized sodium alginate were dissolved in deionized water separately to prepare a 2.5 mg/mL aldehyde dehydrogenase solution and a 1.0 mg/mL oxidized sodium alginate solution. 0.8 mL of the aldehyde dehydrogenase solution and 0.2 mL of the oxidized sodium alginate solution were mixed under ice bath conditions, followed by magnetic stirring for 5 hours to obtain the immobilized aldehyde dehydrogenase nanogel. The particle size detection results, as described in FIG. 7, show that the average particle size is 321 nm.

### Implementation Example 8

Preparation of immobilized lysozyme nanogel: Lysozyme and oxidized sodium alginate were dissolved in deionized water separately to prepare a 2.5 mg/mL lysozyme solution and a 2.0 mg/mL oxidized sodium alginate solution. 0.8 mL of the lysozyme solution and 0.2 mL of the oxidized sodium alginate solution were mixed at room temperature and subjected to a constant temperature reaction at 40° C. for 30 minutes to obtain the immobilized lysozyme nanogel. The particle size detection results, as described in FIG. 8, show that the average particle size is 264 nm.

### Implementation Example 9

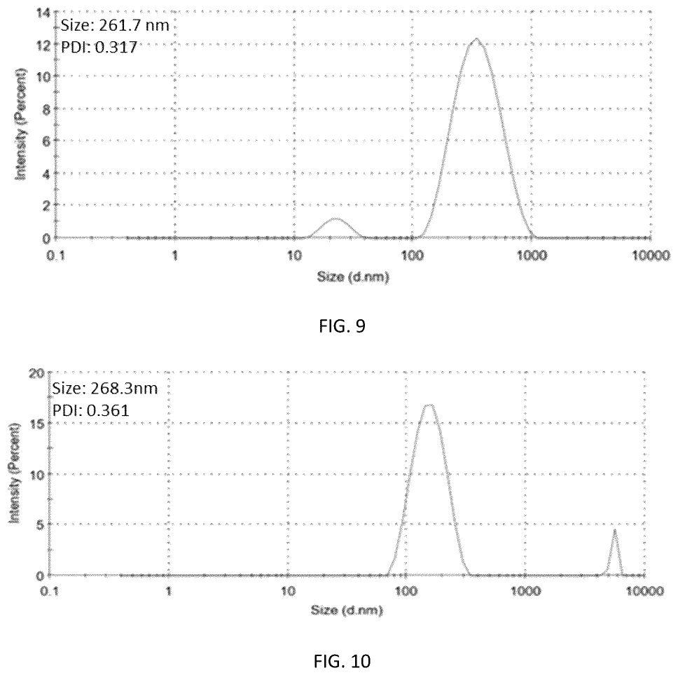

Preparation of immobilized adenosine deaminase nanogel: Adenosine deaminase and oxidized sodium alginate were dissolved in deionized water separately to prepare a 1.0 mg/mL adenosine deaminase solution and a 1.0 mg/mL oxidized sodium alginate solution. 0.8 mL of the adenosine deaminase solution and 0.2 mL of the oxidized sodium alginate solution were mixed under ice bath conditions, followed by magnetic stirring for 6 hours to obtain the immobilized adenosine deaminase nanogel. The particle size detection results, as described in FIG. 9, show that the average particle size is 261 nm.

### Implementation Example 10

Preparation of immobilized papain nanogel: Papain and oxidized sodium alginate were dissolved in deionized water separately to prepare a 2.5 mg/mL papain solution and a 4.0 mg/mL oxidized sodium alginate solution. 0.8 mL of the papain solution and 0.1 mL of the oxidized sodium alginate solution were mixed under ice bath conditions, followed by magnetic stirring for 5 hours to obtain the immobilized papain nanogel. The particle size detection results, as described in FIG. 10, show that the average particle size is 268 nm.

### Implementation Example 11

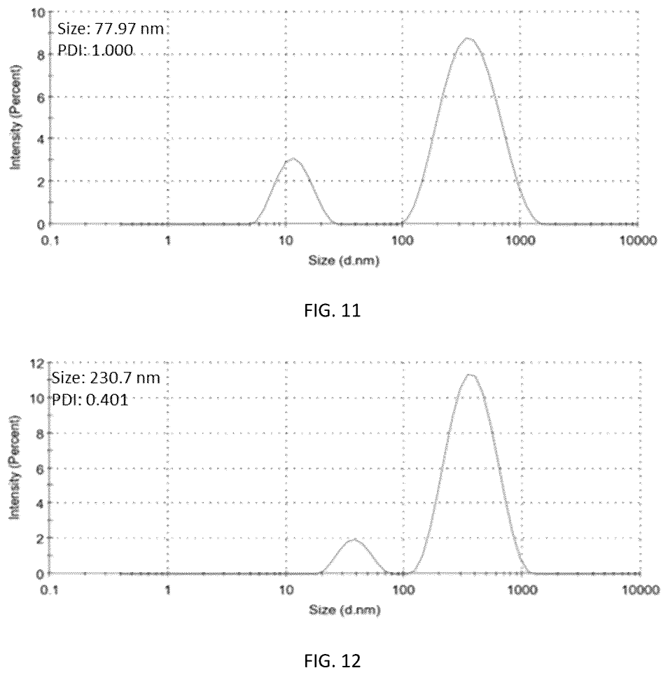

Preparation of immobilized glucose oxidase nanogel: Glucose oxidase and oxidized sodium alginate were dissolved in deionized water separately to prepare a 2.5 mg/mL glucose oxidase solution and a 4 mg/mL oxidized sodium alginate solution. 0.8 mL of the glucose oxidase solution and 0.1 mL of the oxidized sodium alginate solution were mixed under ice bath conditions, followed by magnetic stirring for 5 hours to obtain the immobilized glucose oxidase nanogel. The particle size detection results, as described in FIG. 11, show that the average particle size is 77 nm.

### Implementation Example 12

Preparation of immobilized hyaluronidase nanogel: Hyaluronidase and oxidized sodium alginate were dissolved in deionized water separately to prepare a 2.5 mg/mL hyaluronidase solution and a 4 mg/mL oxidized sodium alginate solution. 0.8 mL of the hyaluronidase solution and 0.1 mL of the oxidized sodium alginate solution were mixed under ice bath conditions, followed by magnetic stirring for 5 hours to obtain the immobilized hyaluronidase nanogel. The particle size detection results, as described in FIG. 12, show that the average particle size is 230 nm.

### Implementation Example 13

Preparation of immobilized collagenase nanogel: Collagenase IV, oxidized sodium alginate, and DV1 were dissolved in deionized water separately to prepare a 1.25 mg/mL collagenase IV solution, a 2 mg/mL oxidized sodium alginate solution, and a 5 mg/mL DV1 solution. 0.8 mL of the collagenase IV solution and 0.1 mL of the oxidized sodium alginate solution were mixed under ice bath conditions, followed by magnetic stirring for 5 hours to obtain the immobilized collagenase nanogel (O-Colase).

### Implementation Example 14

In this example, surface modification of CXCR4 antagonistic peptide was performed on the immobilized collagenase nanogel from Implementation Example 13. This was done to further investigate whether the immobilized enzyme provided by this invention can be used in vivo.

Under stirring, 0.1 mL of DV1 solution was added dropwise to the reaction system of the immobilized collagenase nanogel obtained in Implementation Example 13. The reaction was continued for 1 hour, resulting in the formation of DV1-modified immobilized collagenase nanogel (D#O-Case).

Characterization of D#O-Case in this Implementation:

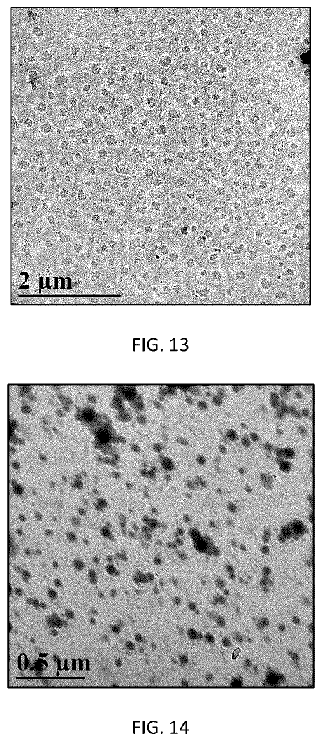

After aspirating 20 μL of the magnetic stirring reaction solution using a pipette gun and allowing it to react for 5 hours, the O-Colase solution and the reaction solution of D#O-Case were obtained. These solutions were then dropped onto a carbon-coated copper grid, excess liquid was removed with filter paper, and the samples were dried at room temperature. Subsequently, the samples were observed under a transmission electron microscope. The results shown in FIG. 13 (O-Colase) and FIG. 14 (D#O-Case) confirm that the size of the D#O-Case prepared in this implementation is 164.1±1.34 nm, with uniform size and good dispersibility.

### Experimental Example 1

Mouse pancreatic cancer Panc02 cells in logarithmic growth phase were collected. The cells were added to the upper chamber of a 24-well Transwell device at a concentration of 5×10⁴ cells/well. The upper chamber contained 100 μL of drug-containing medium, while the lower chamber contained 500 μL of medium containing 100 ng/mL CXCL12. The drug groups included: untreated (Control), DV1, and D#O-Case prepared in Example 14. The concentration of DV1 in the latter two groups was 25 μM. Each drug group underwent three parallel experiments.

After incubating for 24 hours, remove the Transwell chambers, discard the culture medium in the wells, wash twice with calcium-free PBS, fix with methanol for 30 minutes, and air-dry the chambers appropriately.

Stain with 0.1% crystal violet for 20 minutes, gently remove the non-migrated cells from the upper layer using a cotton swab, and wash three times with PBS. Observe and count the migrated cells in three random fields under a 400× magnification microscope.

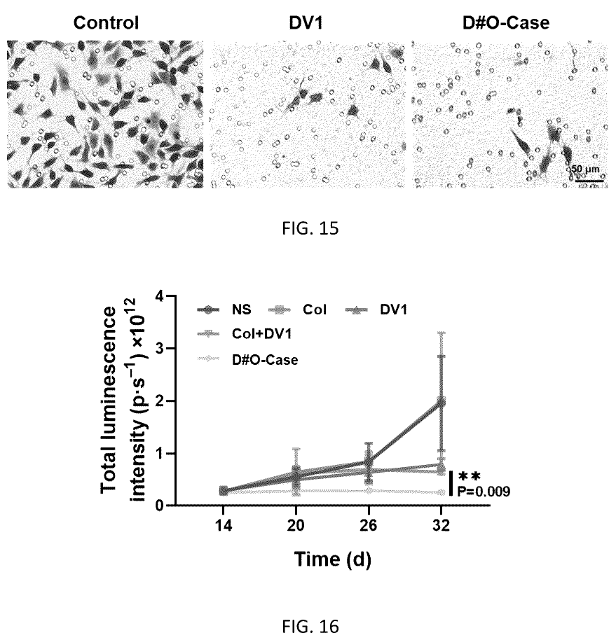

As shown in FIG. 15, the number of migrated cells in the DV1 and D#O-Case treatment groups is essentially the same, and both are significantly less than the untreated group. Therefore, the results indicate that D#O-Case can effectively inhibit tumor cell migration.

### Experimental Example 2 (In Vivo Antitumor Study of D#O-Case)

### 1. Animal Model Establishment

Male C57BL/6 mice aged 9-10 weeks were used to establish an in vivo anti-tumor model. A suspension of pancreatic ductal adenocarcinoma cells (Luc-Panc02 cells) in Matrigel (50%) at a concentration of 1×10⁶ cells per mouse was surgically implanted into the pancreas of the male C57BL/6 mice aged 9-10 weeks. All animal experiments were approved by Shandong University Animal Experiment Ethics Review and the Health Guide for the Care and Use of Laboratory Animals of National Institutes (SYXK(Lu)20200022).

### 2. In Vivo Antitumor Experiment in Animals

14 days after tumor inoculation, bioluminescence imaging was performed on the mice and they were randomly divided into 4 groups: normal saline as a control, collagenase, DV1, DV1+collagenase, and D#O-Case as experimental groups. The mice were treated with drugs intravenously via the tail every 3 days for a total of 6 treatments. Tumor growth was monitored using bioluminescence imaging. At the end of the experiment, the total bioluminescence was used as the statistical data to plot the tumor growth curve. As shown in FIG. 16, D#O-Case effectively inhibits tumor growth.

### Experimental Example 3 (In Vivo Antitumor Study of D#O-Case Combined with CAR-T Cells (CAR-T-D#O-Case))

### 1. Animal Model Establishment

Male NVSG mice aged 9-10 weeks were used to establish an in vivo anti-tumor model. A suspension of Luc-AsPC-1 cells in Matrigel (50%) at a concentration of 1×10⁶ cells per mouse was surgically implanted into the pancreas of male NVSG mice aged 9-10 weeks.

### 2. In Vivo Antitumor Experiment in Animals

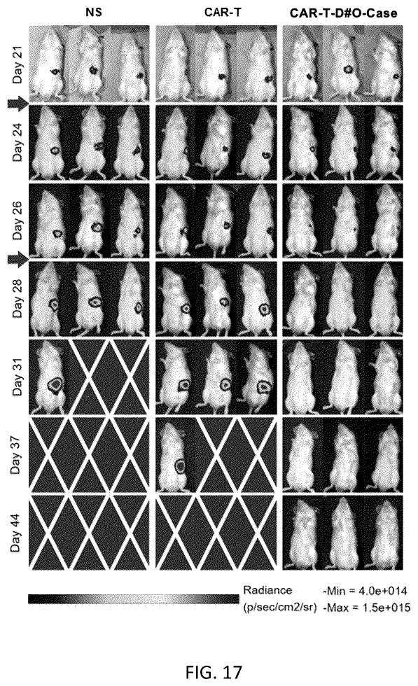

21 days after tumor inoculation, bioluminescence imaging was performed on the mice, and they were randomly divided into 3 groups: normal saline as a control, CAR-T cells (targeting mesothelin), and CAR-T-D#O-Case as the experimental groups. Treatments were administered via tail vein injection on the 21st and 26th days, totaling 2 treatments. Tumor growth was monitored using bioluminescence imaging. As shown in FIG. 17, CAR-T-D#O-Case effectively inhibits tumor growth.

The above description is merely preferred embodiments of the present invention and is not intended to limit the scope of the invention. For those skilled in the art, the present invention can be modified and varied in various ways. Any modifications, equivalent replacements, improvements, etc., made within the spirit and principles of the present invention should be included within the scope of the protection of the present invention.

What is claimed is:

1. A method for preparing immobilized enzyme nanogels, comprising: dissolving oxidized sodium alginate and an enzyme separately in deionized water, mixing uniformly, and obtaining immobilized enzyme nanogels.

2. The method for preparing immobilized enzyme nanogels as claimed in claim 1, wherein the enzyme is one or more selected from the group consisting of oxidoreductases, transferases, hydrolases, lyases, isomerases, synthetases and ligases;
further the enzyme preferably is one or more selected from the group consisting of collagenase, trypsin, deoxyribonuclease I, proteinase K, lactate dehydrogenase, alcohol dehydrogenase, glutamine transferase, aldehyde dehydrogenase, lysozyme, adenosine deaminase, papain, glucose oxidase, and hyaluronidase;
wherein a ratio of the oxidized sodium alginate to the enzyme is 0.01 to 50:1 by weight; a concentration of the oxidized sodium alginate in the deionized water is 1 to 50 mg mL⁻¹.

3. A method for preparing CXCR4 antagonist peptide-modified immobilized matrix-degrading enzyme nanogels, comprising the following steps:
i) dissolving oxidized sodium alginate and a matrix-degrading enzyme separately in deionized water, mixing uniformly, stirring a reaction mixture in an ice bath for a period of time, and obtaining immobilized matrix-degrading enzyme nanogels; and
ii) adding CXCR4 antagonist peptide dropwise to the immobilized matrix-degrading enzyme nanogels while stirring, and obtaining CXCR4 antagonist peptide-modified immobilized matrix-degrading enzyme nanogels.

4. The method for preparing CXCR4 antagonist peptide-modified immobilized matrix-degrading enzyme nanogels as claimed in claim 3, wherein the matrix-degrading enzyme is one or more selected from the group consisting of collagenase, papain, trypsin, tissue proteinase, hyaluronidase, heparinase, and DNAse; the matrix-degrading enzyme is preferably collagenase I-V, the matrix-degrading enzyme further preferably collagenase IV; and
wherein the CXCR4 antagonist peptide is selected from group consisting of DV1, Balixafortide, CTCE-9908, FC131, ALX 40-4C, Motixafortide, CTCE-0214, TC14012, ATI-2341, and derivatives thereof; the CXCR4 antagonist peptide is preferably DV1.

5. The method for preparing CXCR4 antagonist peptide-modified immobilized matrix-degrading enzyme nanogels as claimed in claim 3, wherein the concentration of the matrix-degrading enzyme dissolved in deionized water in the step i) is 0.5 to 5 mg/mL, preferably 1.11 mg/mL;
a ratio of the matrix-degrading enzyme to the oxidized sodium alginate is 1 to 10:1 by weight, preferably 5:1;
the temperature of the ice bath is 0 to 4° C. in the step i); and
the stirring reaction time in the step i) is 4 to 12 hours, preferably 5 hours.

6. The method for preparing CXCR4 antagonist peptide-modified immobilized matrix-degrading enzyme nanogels as claimed in claim 3, wherein in the step ii), the concentration of the CXCR4 antagonist peptide is 0.5 to 5 mg/mL, preferably 1 mg/mL;
in the step ii), a ratio of the CXCR4 antagonist peptide to the matrix-degrading enzyme from the step i) is 1:0.1 to 10 by weight, preferably 1:2;
a reaction time in the step ii) is 0.1 to 5 hours, preferably 1 hour; and
a reaction in the step ii) is carried out in the ice bath at temperature of 0 to 4° C.

7. The method for preparing CXCR4 antagonist peptide-modified immobilized matrix-degrading enzyme nanogels as claimed in claim 4, wherein the CXCR4 antagonist peptide-modified immobilized matrix-degrading enzyme nanogels is prepared as an anti-tumor drug, characterized by having at least one or more of following applications:
a) reducing density of tumor extracellular matrix;
b) increasing infiltrating lymphocytes in tumors;
c) promoting migration of T cells to tumors;
d) inhibiting tumor metastasis;
e) increasing an effect of CAR-T cell solid tumor;
wherein c), d) and d) are related to CXCR4.

8. A composition of an anti-tumor drug, comprising the CXCR4 antagonist peptide-modified immobilized matrix-degrading enzyme nanogels as claimed in claim 4.
